(19) United States
(12) Patent Application Publication
(10) Pub. No.: US 2016/0220466 A1
(43) Pub. Date: Aug. 4, 2016

(54) HAIR TREATMENT COMPOSITION

(71) Applicant: Lonza Ltd., Visp (CH)

(72) Inventors: Paul Hanselmann, Visp (CH); Florencio Zaragoza Doerwald, Visp (CH)

(21) Appl. No.: 15/010,180

(22) Filed: Jan. 29, 2016

Related U.S. Application Data
(60) Provisional application No. 62/110,010, filed on Jan. 30, 2015.

(51) Int. Cl. A61K 8/49; A61K 8/37; A45D 7/06; A61Q 5/06

(52) U.S. Cl. CPC A61K 8/4973; A61Q 5/06; A61K 8/37; A45D 7/06; A61K 2800/40

(57) ABSTRACT

A hair treatment composition is described that contains an acetoacetylated compound. The acetoacetylated compound, for instance, can be a bis acetoacetylated compound. In one embodiment, for instance, the acetoacetylated compound comprises a bis acetoacetate or a bis acetoacetamide. In one embodiment, the hair treatment composition can be used to straighten hair. The composition is also well suited for decreasing hair frizz. The acetoacetylated compound is capable of crosslinking hair fibers upon the application of heat.

### RELATED APPLICATIONS

The present application is based on and claims priority to U.S. Provisional Patent application Ser. No. 62/110,010, which was filed on Jan. 30, 2015, and which is incorporated herein by reference.

### BACKGROUND

Throughout history, men, women and children have spent significant amounts of time and energy in styling their hair for different reasons. Having an attractive or aesthetically pleasing hairstyle, for instance, not only improves the appearance of the individual but also suggests that the individual is well groomed. Hairstyles are also used to keep up with current fashions or even start a fashion trend. Various different cultural and practical considerations influence hairstyles.

There are many different techniques and methods that can be used in order to style one's hair. The most rudimentary method for styling hair is simply to comb or brush the hair in a certain way. Those in the past have also colored their hair for a certain desired look. In addition to the above, in the past, various different techniques have been used in order to increase the amount of curls in one's hair or to place hair curls at certain locations and to incorporate curls into the hair in a manner that makes the curl somewhat permanent for an extended length of time.

Another technique that is particularly popular today is to, instead of curling the hair, straightening the hair. Hair straightening is a hairstyling technique that involves flattening and straightening the hair in order to give it a smooth, streamlined and sleek appearance. Hair straightening can be accomplished using different methods. In the past, for instance, various gels and other similar materials were placed in the hair in order to give the hair a straightened appearance.

In the more recent past, hair straightening has been accomplished using heat alone or in combination with chemical auxiliaries. One type of hair straightening treatment is referred as the “Brazilian” hair straightening technique, that is sometimes referred to keratin straightening. During Brazilian hair straightening, the hair is straightened by sealing a liquid keratin solution into the hair with a hair iron. Brazilian hair straightening treatments eliminate frizz and cause the hair to straighten for at least several weeks to several months. Unfortunately, the method is typically used with formaldehyde or a chemical compound that produces formaldehyde when exposed to heat. Although the use of formaldehyde is effective in treating damaged hair and eliminating curls, the use of formaldehyde is highly regulated in some countries and somewhat disfavored. Thus, there is increasing pressure in order to find a replacement to formaldehyde in hair straightening and other hair treatment processes.

For instance, in the past, the use of formaldehyde has been replaced by using glyoxylic acid derivatives. In another alternative, those skilled in the art have suggested using alpha-keto acids as formaldehyde replacements.

Further improvements, however, are still needed in finding safe and effective cosmetic compositions that may be used to straighten hair.

### SUMMARY

The present disclosure is generally directed to a hair treatment composition and to a process for applying the hair treatment composition to keratin fibers, such as hair or fur. In one embodiment, the hair treatment composition can be used to straighten hair. Through the process of the present disclosure, hair can be straightened temporarily, but for a significant amount of time, such as at least a few days to several months.

In one embodiment, the present disclosure is directed to a hair treatment composition comprising an acetoacetylated compound. In accordance with the present disclosure, an acetoacetylated compound is selected that is capable of crosslinking keratin fibers. For instance, in one embodiment, the acetoacetylated compound may be capable of crosslinking with NH₂ groups or OH groups.

One embodiment of an acetoacetylated compound that may be used in accordance with the present disclosure is as follows:

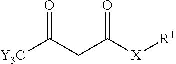

wherein Y is hydrogen or a halogen, such as fluorine, chlorine, bromine, or iodine, X comprises oxygen, nitrogen, or sulfur, and R¹ is any suitable group capable of forming a covalent bond with keratin or with other acetoacetyl compounds. R¹, for instance, may comprise one or more acetoacetylated groups, may contain one or more thiol groups, may contain one or more hydroxyl groups, may contain one or more carbonyl groups, may contain one or more vinyl groups, may contain one or more epoxy groups, may contain one or more acetal groups, or may contain a combination of the above.

In one particular embodiment, the acetoacetylated compound comprises a bis acetoacetylated compound. For example, the compound can have the following formula:

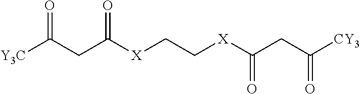

wherein X comprises N, O, or S, and Y comprises hydrogen or halogen. When X comprises oxygen and Y is hydrogen, the acetoacetylated compound is acetoacetylated ethylene glycol. When X comprises nitrogen and Y is hydrogen, on the other hand, the acetoacetylated compound comprises acetoacetylated ethylene diamine.

In addition to the acetoacetylated compound, the hair treatment composition may comprise various other components. The hair treatment composition, for instance, can be in the form of a fluid, such as a liquid or gel that may contact hair. For instance, the hair treatment composition may comprise a spray, a foam, a lotion, a cream, a mousse, an aerosol, a shampoo, a serum, an oil, or a conditioner.

In one embodiment, the hair treatment composition contains a liquid carrier for the acetoacetylated compound. The liquid carrier, for instance, may comprise water, an alcohol, or mixtures thereof. The hair treatment composition may also contain a surfactant and/or a viscosity modifier. The viscosity modifier may comprise a polyol, such as polyethylene glycol.

In one embodiment, the acetoacetylated compound can be present in the hair treatment composition in an amount from about 0.01% by weight to about 80% by weight, such as from about 0.01% by weight to about 25% by weight. In one embodiment, the acetoacetylated compound is present in the composition in an amount greater than about 0.1% by weight, such as in an amount greater than about 0.5% by weight, such as in an amount greater than about 1% by weight. The acetoacetylated compound can be present in the composition in an amount less than about 25% by weight, such as in an amount less than about 20% by weight, such as in an amount less than about 15% by weight, such as in an amount less than about 10% by weight, such as in an amount less than about 7% by weight.

The hair treatment composition of the present disclosure may have many different uses and purposes. In general, the hair treatment composition of the present disclosure can be used to style hair. Alternatively, the composition may be used to repair damaged hair by strengthening or stabilizing the fiber. For instance, the hair treatment composition of the present disclosure may be used to repair hair frizz and/or split ends. When repairing damaged hair, the hair treatment composition may be simply applied to the hair and allowed to dry. In one embodiment, a blow dryer may be used to provide some heat. In an alternative embodiment, a heated implement may be used, such as a heated flat iron.

In an alternative embodiment, the hair treatment composition can be used to straighten hair. The process includes contacting keratin fibers, such as hair, with the hair treatment composition described above. After being contacted with the hair treatment composition, heat is applied in an amount sufficient to cause the hair fibers to crosslink. In one embodiment, for instance, the hair fibers crosslink while being heated to a temperature of from about 40° C. to about 250° C. The temperature, for instance, may be greater than about 40° C., such as greater than about 50° C., such as greater than about 60° C., such as greater than about 70° C., such as greater than about 80° C., such as greater than about 100° C., such as greater than about 110° C., such as greater than about 120° C. The temperature is generally less than about 200° C., such as less than about 180° C. Of particular advantage, crosslinking can occur at relatively low temperatures, such as less than about 160° C., such as less than about 140° C. Heat can be applied to the hair using various different methods and techniques. For instance, in one embodiment, a straightening iron may be used. In an alternative embodiment, the hair can be contacted with heated air, such as air from a blow dryer.

Other features and aspects of the present disclosure are discussed in greater detail below.

### BRIEF DESCRIPTION OF THE DRAWINGS

FIGS. 1 and 2 are graphical representations of results obtained in Example numbers 1 and 2 below.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to a hair treatment composition that can be used for hair straightening. Hair straightening refers to the process of reducing the number of curls in hair and/or reducing their radius of curvature of curls within the hair. In one embodiment, hair straightening can be used to completely eliminate curls.

In addition to hair straightening, the hair treatment composition of the present disclosure has numerous other uses and applications. For instance, in one embodiment, the hair treatment composition can be used to repair damaged hair. For example, the hair treatment composition can be used to treat hair frizz and/or treat split ends. In still another embodiment, the hair treatment composition can be used as a styling agent.

Keratin fibers, such as hair (which includes fur), are made from keratin, which is a protein. Hair can have various different properties and characteristics depending on its underlying chemistry. Hair, for instance, can be coarse or fine and can be relatively straight or curly. Hair gets much of its structure from two types of chemical bonds in keratin. The bonds include noncovalent hydrogen bonds and covalent disulfide bonds. Applying heat to hair can temporarily straighten hair by rearranging the hydrogen bonds. Simply applying heat to hair, however, produces a straightened appearance that may only last less than a day and will be reversed if contacted with water or even humid air.

The present disclosure is directed to a hair treatment composition that may be used in conjunction with heat for straightening hair for a longer period of time. Hair straightened according to the present disclosure, for instance, can maintain its shape and style for periods of time of greater than one week, such as greater than two weeks, such as greater than four weeks, such as greater than eight weeks, such as greater than ten weeks, such as even greater than twelve weeks.

In certain embodiments, the straightened hair can maintain its form and shape for as long as necessary until the hair is subjected to a number of washes. For instance, in one embodiment, hair straightened according to the present disclosure can maintain its shape and style, for instance, for more than 2 shampoo washes, such as more than 4 shampoo washes, such as more than 6 shampoo washes, such as more than 8 shampoo washes, such as more than 10 shampoo washes, such as even more than 12 shampoo washes. In one embodiment, the straightened hair can maintain its shape and style for up to about 15 shampoo washes.

The hair treatment composition of the present disclosure, for instance, contains an acetoacetylated compound that acts as a cross-linking reagent when contacted with hair. For example, the acetoacetylated compound may crosslink with NH₂ groups and/or OH groups present on keratin fibers.

An acetoacetylated compound is a compound that has been formed through acetoacetylation. Acetoacetylation refers to a chemical reaction, in which an acidic X—H group, X being oxygen, nitrogen, sulfur, or carbon, is converted into a X—C(═O)—CH₂—C(═O)—CH₃ group. Such a reaction can be accomplished by treatment of the acidic X—H group with diketene (4-methyleneoxetan-2-one) or a synthetic equivalent thereof, such as e.g. 2,2,6-trimethyl-4H-1,3-dioxin-4-one. In addition tert.-butyl acetoacetate is another possible reagent for acetoacetylation. In the present disclosure, ‘acetoacetylated compounds’ also refers to compounds containing partially halogenated acetoacetyl groups, such as, for instance, trifluoroacetoacetyl groups or trichloroacetoacetyl groups.

In general, an acetoacetylated compound in accordance with the present disclosure comprises any acetoacetylated compound capable of crosslinking to keratin fibers and/or other acetoacetylated compounds. In one embodiment, the acetoacetylated compound may have the following chemical formula:

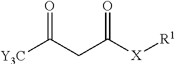

wherein Y, X, and R¹ are as defined above.

In one embodiment, the acetoacetylated compound may comprise a bis acetoacetylated compound (i.e., a compound with two acetoacetyl groups). For instance, the acetoacetylated compound may comprise a polyol acetoacetylated two times, a diamine acetoacetylated two times, or an aminoalcohol acetoacetylated two times. Specific suitable substrates for acetoacetylation according to the present invention include, but are not limited to, ethylene glycol, 1,2-propylene glycol, 1,3-propylene glycol, 1,2-butylene glycol, 1,4-butylene glycol, 2,2-dimethyl-1,3-propylene glycol, 2-aminoethanol, 3-amino-1-propanol, glycerol, diethylene glycol (HO—CH₂—CH₂—O—CH₂—CH₂—OH), triethylene glycol (HO—(CH₂—CH₂—O)₃—H, 2-methyl-2-amino-1,3-propanediol, 2-amino-tris-(hydroxymethyl)methan, trimethylolpropane, trimethylolethane, polyvinylalkolhol, or 2-aminoethanethiol.

In addition to twofold acetoacetylated compounds, various other poly(acetoacetylated) compounds may be used. For instance, the acetoacetylated compound may comprise a tris acetoacetylated compound or a tetrakis acetoacetylated compound. The poly(acetoacetylated) compounds can be per- or partially acetoacetylated glucose, other per- or partially acetoacetylated carbohydrates, or other per- or partially acetoacetylated polyols. Other polyols include glycerol, sorbitol, mannitol, meglumine, ascorbic acid, pentaerythritol, and/or tris(hydroxymethyl)aminomethane. Any such poly(acetoacetylated) compounds may be used with the exception of tertiary amines.

In one embodiment, the acetoacetylated compound has the following formula:

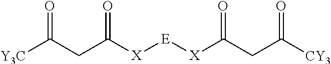

wherein each X and Y are independently as defined above, and E comprises any suitable group that does not interfere with the crosslinking reaction. In one embodiment, for instance, E can comprise any straight or branched alkylene chain (a saturated, hydrocarbon-derived diradical), unsaturated or saturated alkylene groups, or alkylene chains interrupted by ether linkages. In one particular embodiment, E comprises an alkylene group having a carbon chain length of from about 2 carbon atoms to about 6 carbon atoms.

When X comprises oxygen, the acetoacetylated compound can be considered a bis acetoacetate or an acetoacetylated polyol. When X comprises nitrogen, on the other hand, the acetoacetylated compound may be considered a bis acetoacetamide.

In one particular embodiment, the acetoacetylated compound may comprise one of the following:

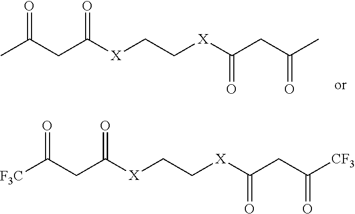

wherein X═O or NR¹¹ and R¹¹ comprises hydrogen or a C₁₋₅ alkyl group. In one additional specific embodiment, the acetoacetylated compounds may be N,N′-bis(acetoacetyl)piperazine and N,N′-bis(trifluoroacetoacetyl)piperazine.

In one embodiment, the acetoacetylated compound may include a thiol group. Some examples are as follows:

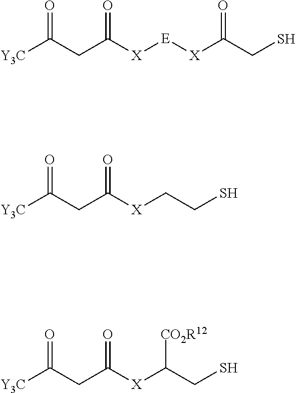

wherein each X and Y are independently as defined above, and R¹² comprises C₁₋₆ alkyl.

In still other embodiments, the acetoacetylated compound may comprise acetal groups or vinyl groups. Examples include the following:

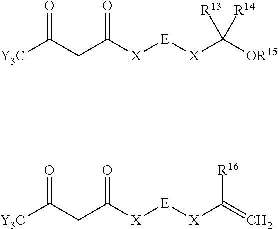

wherein E and each X and Y are independently as defined above, R¹³, R¹⁴, and R¹⁶ independently represent hydrogen or C₁₋₄ alkyl, and R¹⁵ is acetyl or C₁₋₄ alkyl.

In one additional embodiment, the acetoacetylated compound may comprise epoxy groups. Examples include the following:

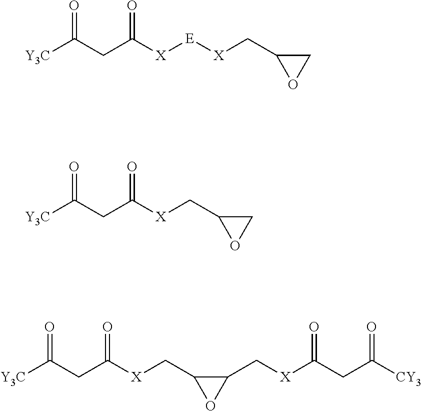

wherein E and each X and Y are independently as defined above.

Of particular advantage, the properties of the acetoacetylated compound can be controlled by controlling the molecular structure. For example, modifying the R and X groups in the above formula allows various characteristics of the compound to be changed such as whether the compound is a liquid or solid at room temperature, whether the compound is lipophilic, whether the compound is hydrophilic or hydrophobic, and the like. Also of advantage, the acetoacetylated compounds of the present disclosure may have a very pleasant odor which may alleviate the necessity to include a fragrance in the hair treatment composition.

It is unknown how the acetoacetylated compounds of the present disclosure repair damaged hair and/or straighten hair. It is believed that the acetoacetylated compounds of the present disclosure fuse additional amino acids and/or proteins from keratin to the hair fiber during a cross-linking reaction. For instance, the acetoacetylated compound may form crosslinks between two opposing NH₂ groups.

Although unknown, it is believed that the acetoacetylated compounds may form imine bonds and enamine bonds with the hair fibers.

After the hair undergoes the above crosslinking reaction, the hair will maintain a straightened configuration. For instance, after the hair fiber is pulled straight and heated, the fiber goes straight and the straightened structure is stabilized due to the crosslinking reaction. Further, in addition to the crosslinking reaction shown above, various other crosslinking can occur. For instance, the acetoacetylated compounds may undergo self-crosslinking reactions during the process.

The hair treatment composition of the present disclosure can contain one or more acetoacetylated compounds in amounts generally from about 0.01% by weight to about 90% by weight. In general, the acetoacetylated compounds are present in the hair treatment composition in an amount less than about 80% by weight, such as in an amount less than about 70% by weight, such as in an amount less than about 60% by weight, such as in an amount less than about 50% by weight, such as in an amount less than about 40% by weight, such as in an amount less than about 30% by weight, such as in an amount less than about 20% by weight, such as in an amount less than about 15% by weight, such as in an amount less than about 10% by weight. One or more acetoacetylated compounds can be present in the hair treatment composition in an amount greater than about 0.5% by weight, such as in an amount greater than about 1% by weight, such as in an amount greater than about 2% by weight.

In addition to one or more acetoacetylated compounds, the hair treatment composition can contain various other components depending upon the particular application. The one or more components can be combined with the acetoacetylated compound to form various different products. For instance, the product may comprise a hair straightening spray. In other embodiments, however, the hair treatment composition may comprise a shampoo, a conditioner, a lotion, a mousse, a gel, a cream, or any other suitable cosmetic product.

In one embodiment, the hair treatment composition contains a liquid carrier for the acetoacetylated compound. The liquid carrier, for instance, may comprise water and/or an alcohol. In general, any suitable liquid carrier can be used that does not interfere with the ability of the acetoacetylated compound to undergo a crosslinking reaction. The liquid carrier can be present in the composition in an amount from 5% by weight to about 99% by weight. In general, the liquid carrier is present in an amount greater than about 20% by weight, such as in an amount greater than about 30% by weight, such as in an amount greater than about 40% by weight, such as in an amount greater than about 50% by weight, such as in an amount greater than about 60% by weight. The liquid carrier can be present in an amount less than about 95% by weight, such as in an amount less than about 90% by weight, such as in an amount less than about 80% by weight, such as in an amount less than about 70% by weight.

In one embodiment, the hair treatment composition may contain a viscosity modifier. The viscosity modifier may also serve as an emollient or thickener. In one embodiment, the viscosity modifier may comprise a polyol, which include diols. Polyols that may be used include glycerol, ethylene glycol, propylene glycol, butylene glycol, sorbitol, caprylyl glycol, 1,3-butane diol, hexylene glycol, isoprene glycol, xylitol, and the like. Other viscosity modifiers include ethylhexyl polmitate, a triglyceride, or a fatty acid ester such as cetearyl isononanoate or cetyl palmitate. In one embodiment, the viscosity modifier may comprise a dimethicone. In still another embodiment, the viscosity modifier may comprise a polymeric glycol. Examples of polymeric glycols include polypropylene glycol and/or polyethylene glycol. In one embodiment, for instance, polyethylene glycol 200 or polyethylene glycol 400 may be used.

When present, the viscosity modifier can be contained in the hair treatment composition in an amount greater than about 0.05% by weight, such as in an amount greater than about 0.5% by weight, such as in an amount greater than about 1% by weight. The viscosity modifier is generally present in an amount less than about 25% by weight, such as in an amount less than about 20% by weight, such as in an amount less than about 15% by weight, such as in an amount less than about 10% by weight.

In one embodiment, the hair treatment composition can contain one or more surfactants. In general, any suitable surfactant may be used that does not destroy the stability of the composition and/or interfere with the crosslinking potential of the acetoacetylated compound. The surfactant may comprise an anionic surfactant, a zwitterionic surfactant, a nonionic surfactant, a cationic surfactant, or mixtures thereof.

In one embodiment, for instance, an anionic surfactant may be present in the composition. For instance, the surfactant may comprise a water-soluble salt of a fatty alcohol derivative. For instance, the surfactant may comprise a fatty alcohol sulfate that has a carbon chain length of from about 10 carbon atoms to about 14 carbon atoms. The fatty alcohol salt may comprise a sodium salt, a potassium salt, an ammonium salt, a diethanol ammonium salt, a triethanol ammonium salt, or mixtures thereof. In one particular embodiment, the surfactant may comprise sodium lauryl sulfate.

As a general matter, anionic surfactants are exemplified by the alkali metal salts of organic sulfuric reaction products having in their molecular structure an alkyl radical containing from 8 to 22 carbon atoms and a sulfonic acid or sulfuric acid ester radical (included in the term alkyl is the alkyl portion of higher acyl radicals). Preferred alkyl sulfates are sodium coconut oil fatty acid monoglyceride sulfate and sulfonates and those obtained by sulfating higher alcohols, i.e., those containing C₈ to C₁₈ carbon atoms. Other examples are sodium or potassium salts of sulfuric acid esters of the reaction product of 1 mole of a higher fatty alcohol (e.g., tallow or coconut oil alcohols) and 1 to 12 moles of ethylene oxide; sodium or potassium salts of alkyl phenol ethylene oxide ether sulfate with 1 to 10 units of ethylene oxide per molecule and in which the alkyl radicals contain from 8 to 12 carbon atoms, sodium alkyl glyceryl ether sulfonates; the reaction product of fatty acids having from 10 to 22 carbon atoms esterified with isethionic acid and neutralized with sodium hydroxide; and water-soluble salts of condensation products of fatty acids with sarcosine.

In another embodiment, the surfactant may comprise a quaternary compound. Examples of quaternary compounds, for instance, include cetyltrimonium chloride, behenyltrimonium chloride, tetramethylammonium chloride, tetraethylammonium chloride, octyltrimethylammonium chloride, cocotrimethylammonium chloride, and the like.

In one embodiment, the composition may contain a zwitterionic surfactant. For instance, one example of a zwitterionic surfactant is cocamidopropyl betaine. Other alkylamidopropyl betaines may also be used that have alkyl groups derived from fatty acids having a carbon length of from about 10 carbon atoms to about 16 carbon atoms.

Additional zwitterionic surfactants which can be used are substituted imidazolines. Such surfactants include sodium cocoamphoacetate and disodium cocoamphodiacetate. In addition to, or in place of, the coco-derived imidazolines, one can use similar structures prepared from C₁₀ to C₁₆ fatty acids. The active content of imidazoline surfactant, either as a single reagent or as a blend, runs from about 1.5% to about 2.5%.

Zwitterionic surfactants are exemplified by derivatives of aliphatic quaternary ammonium, phosphonium, and sulfonium compounds, in which the aliphatic radicals are straight chain or branched, and wherein one of the aliphatic substituents contains from about 8 to 18 carbon atoms and one contains an anionic water-solubilizing group, e.g., carboxyl, sulfonate, sulfate, phosphate, or phosphonate. A general formula for these compounds is:

(R³)ₓR²Y⁽⁺⁾—CH₂—R⁴—Z⁽⁻⁾

wherein R² is an alkyl, alkenyl, or hydroxyalkyl radical of from about 8 to about 18 carbon atoms. They may contain up to about 10 ethylene oxide moieties and up to 1 glyceryl moiety. Y is a nitrogen, phosphorus, or sulfur atom; R³ is an alkyl or monohydroxyalkyl group containing 1 to about 3 carbon atoms; X is 1 when Y is a sulfur atom and 2 when Y is a nitrogen or phosphorus atom; R⁴ is an alkylene or hydroxyalkylene of from about 1 to about 4 carbon atoms; and Z is a carboxylate, sulfonate, sulfate, phosphonate, or phosphate group.

Specific examples of zwitterionic surfactants include the following:
4-[N,N-di(2-hydroxyethyl)-N-octadecylammonio]-butane-1-carboxylate;
5-[S-3-hydroxypropyl-S-hexadecylsulfonio]-3-hydroxypentane-1-sulfate;
3-[P,P-diethyl-P-3,6,9-trioxatetradecoxylphosphonio]-2-hydroxyl-propane-1-phosphate;
3-[N,N-dipropyl-N-3-dodecoxy-2-hydroxypropylammonio]-propane-1-phosphonate;
3-(N,N-dimethyl-N-hexadecylammonio)propane-1-sulfonate;
3-(N,N-dimethyl-N-hexadecylammonio)-2-hydroxypropane-1-sulfonate;
4-[N,N-di(2-hydroxyethyl)-N-(2-hydroxydodecyl)ammonio]-butane-1-carboxylate;
3-[S-ethyl-S-(3-dodecoxy-2-hydroxypropyl)sulfonio]-propane-1-phosphate;
3-[P,P-dimethyl-P-dodecylphosphonio]-propane-1-phosphonate; and
5-[N,N-di(3-hydroxypropyl)-N-hexadecylammonio]-2-hydroxypentane-1-sulfate.

Examples of other betaines useful herein include the high alkyl betaines such as:
coco dimethyl carboxymethyl betaine,
lauryl dimethyl carboxymethyl betaine,
lauryl dimethyl alpha-carboxyethyl betaine,
cetyl dimethyl carboxymethyl betaine,
lauryl bis-(2-hydroxyethyl)carboxymethyl betaine,
stearyl bis-(2-hydroxypropyl)carboxymethyl betaine,
oleyl dimethyl gamma-carboxypropyl betaine,
lauryl bis-(2-hydroxypropyl)alpha-carboxyethyl betaine, etc.

The sulfobetaines may be represented by:
coco dimethyl sulfopropyl betaine,
stearyl dimethyl sulfopropyl betaine,
lauryl dimethyl sulfoethyl betaine,
lauryl bis-(2-hydroxyethyl)sulfopropyl betaine and the like. Amido betaines and amidosulfo betaines, wherein the RCONH(CH₂)₃ radical is attached to the nitrogen atom of the betaine may also be present.

Examples of amphoteric surfactants which can be used in the compositions are derivatives of aliphatic secondary and tertiary amines in which the aliphatic radical is straight chain or branched and wherein one of the aliphatic substituents contains from about 8 to about 18 carbon atoms and one contains an anionic water-solubilizing group, e.g., carboxy, sulfonate, sulfate, phosphate, or phosphonate. Examples of these compounds are sodium 3-dodecylaminopropionate, sodium 3-dodecylaminopropane sulfonate, and N-alkyltaurines such as the reaction product of dodecylamine and sodium isethionate, and N-higher alkyl aspartic acids.

Nonionic surfactants may also be present in the composition alone or in combination with the other surfactants. Examples of nonionic surfactants that may be used include linear alcohol alkoxylates, such as linear alcohol ethoxylates, propoxylated fatty alcohols, and polyoxyethylenecetyl ethers.

Other nonionic surfactants include the following:
Long chain tertiary amine oxides corresponding to the following general formula:

R⁵R⁶R⁷N⁽⁺⁾—O⁽⁻⁾

wherein R⁵ contains an alkyl, alkenyl or monohydroxy alkyl radical of from about 8 to about 18 carbon atoms, up to about 10 ethylene oxide moieties, and up to 1 glyceryl moiety, and R⁶ and R⁷ independently contain from 1 to about 3 carbon atoms and up to about 1 hydroxy group, e.g., methyl, ethyl, propyl, hydroxyethyl, or hydroxypropyl radicals. Examples of amine oxides suitable for use in this invention include:
dimethyldodecylamine oxide,
oleyldi(2-hydroxyethyl)amine oxide,
dimethyloctylamine oxide,
dimethyldecylamine oxide,
dimethyltetradecylamine oxide,
3,6,9-trioxaheptadecyldiethylamine oxide,
di(2-hydroxyethyl)tetradecylamine oxide,
2-dodecoxyethyldimethylamine oxide,
3-dodecoxy-2-hydroxypropyldi(3-hydroxypropyl)amine oxide, and
dimethylhexadecylamine oxide.

Long chain tertiary phosphine oxides corresponding to the following general formula:

R⁸R⁹R¹⁰P═O

wherein R⁸ contains an alkyl, alkenyl or monohydroxyalkyl radical ranging from 8 to 18 carbon atoms in chain length, up to about 10 ethylene oxide moieties, and up to 1 glyceryl moiety and R⁹ and R¹⁰ are each alkyl or monohydroxyalkyl groups containing from 1 to 3 carbon atoms. Examples of suitable phosphine oxides are:
dodecyldimethylphosphine oxide,
tetradecyldimethylphosphine oxide,
tetradecylmethylethylphosphine oxide,
3,6,9-trioxaoctadecyldimethylphosphine oxide,
cetyldimethylphosphine oxide,
3-dodecoxy-2-hydroxypropyldi(2-hydroxyethyl)phosphine oxide,
stearyldimethylphosphine oxide,
cetylethylpropylphosphine oxide,
oleyldiethylphosphine oxide,
dodecyldiethylphosphine oxide,
tetradecyldiethylphosphine oxide,
dodecyldipropylphosphine oxide,
dodecyldi(hydroxymethyl)phosphine oxide,
dodecyldi(2-hydroxyethyl)phosphine oxide,
tetradecylmethyl-2-hydroxypropylphosphine oxide,
oleyldimethylphosphine oxide, and
2-hydroxydodecyldimethylphosphine oxide.

Long chain dialkyl sulfoxides containing one short chain alkyl or hydroxy alkyl radical of 1 to about 3 carbon atoms (usually methyl) and one long hydrophobic chain which contains alkyl, alkenyl, hydroxy alkyl, or keto alkyl radicals containing from about 8 to about 20 carbon atoms, up to about 10 ethylene oxide moieties, and up to 1 glyceryl moiety. Examples include: octadecyl methyl sulfoxide, 2-ketotridecyl methyl sulfoxide, 3,6,9-trioxaoctadecyl 2-hydroxyethyl sulfoxide, dodecyl methyl sulfoxide, oleyl 3-hydroxypropyl sulfoxide, tetradecyl methyl sulfoxide, 3-methoxytridecyl methyl sulfoxide, 3-hydroxytridecyl methyl sulfoxide, 3-hydroxy-4-dodecoxybutyl methyl sulfoxide.

The polyethylene oxide condensates of alkyl phenols, e.g., the condensation products of alkyl phenols having an alkyl group containing from about 6 to 12 carbon atoms in either a straight chain or branched chain configuration, with ethylene oxide, the said ethylene oxide being present in amounts equal to 10 to 60 moles of ethylene oxide per mole of alkyl phenol. The alkyl substituent in such compounds may be derived from polymerized propylene, diisobutylene, octane, or nonane.

The condensation product of straight or branched chain aliphatic alcohols having from 8 to 18 carbon atoms with ethylene oxide, e.g., a coconut alcohol ethylene oxide condensate having from 10 to 30 moles of ethylene oxide per mole of coconut alcohol, the coconut alcohol fraction having from 10 to 14 carbon atoms.

When present, one or more surfactants can be contained in the hair treatment composition in an amount greater than about 0.05% by weight, such as in an amount greater than about 0.5% by weight, such as in an amount greater than about 1% by weight. The surfactant is generally present in an amount less than about 30% by weight, such as in an amount less than about 20% by weight, such as in an amount less than about 15% by weight, such as in an amount less than about 10% by weight.

In addition to one or more of the above components, the hair treatment composition of the present disclosure can contain various other ingredients. For instance, the composition may contain a silicone, a dye, a fragrance, a preservative, a buffering or pH control agent, and the like. The composition can be formulated to include one or more of the above described components.

Preservatives include dimethyl dimethylolhydantoin (DMDMH), DMDMH/iodopropynyl-butyl carbamate, benzyl alcohol, methyl paraben, propyl paraben and imidazolidinyl urea. One or more preservatives can be present in the composition in an amount from about 0.1% to about 5% by weight, such as from about 0.5% to about 2% by weight.

One or more fragrances and/or dyes may also be added to the composition. As explained above, however, the acetoacetylated compound may have a pleasant odor thus making the presence of a fragrance optional. Fragrances and dyes are generally present in the composition in an amount of from about 0.01% to about 5% by weight, such as from about 0.05% to about 2% by weight.

In one embodiment, the hair treatment composition can further contain a polyalkylene glycol fatty acid derivative, which may aid in straightening hair. Suitable polyethylene glycol mono stearates include PEG mono stearate having an average molecular weight of from about 200 to about 6,000 Daltons, with a more preferred weight from about 200 to about 2,000 Daltons and a most preferred PEG mono stearate from about 200 to about 1,000 Daltons. Commercially available PEG mono stearate materials include PEG-200 mono stearate, PEG-400 mono stearate, PEG-600 mono stearate, PEG-1,000 mono stearate, PEG-4,400 mono stearate and PEG-6,000 mono stearate. When present, the polyalkylene glycol fatty acid derivative may be present in the composition in an amount from about 0.5% to about 20% by weight. For instance, the polyalkylene glycol fatty acid derivative can be present in an amount greater than about 2% by weight, such as in an amount greater than about 3% by weight, and generally in an amount less than about 15% by weight, such as in an amount less than about 10% by weight.

As described above, the hair treatment composition of the present disclosure can be in various different forms. For instance, the composition can comprise a liquid, gel or mousse. In general, the composition can be in any form capable of being applied to one's hair. In one particular embodiment, the hair treatment composition comprises a liquid that can be sprayed onto the hair.

The hair treatment composition of the present disclosure can be applied to the hair for various different reasons. For instance, in one embodiment, the hair treatment composition may be used as a styling spray or styling gel. Alternatively, the hair treatment composition may be used to repair damaged hair. For instance, the hair treatment composition may be used to reduce hair frizz and/or repair split ends. When being used to repair hair, the addition of heat may not be necessary. For instance, the hair treatment composition may be applied to the hair and then dried. If desired, heat may be supplied by using a blow dryer or curling iron.

Alternatively, the hair treatment composition may be used in order to straighten hair. Various different methods and techniques may be used in order to apply the composition to hair in the process of straightening the hair. In one embodiment, for instance, hair can be contacted with the hair treatment composition. The treated hair is then heated to a temperature sufficient for the crosslinking action to occur. The presence of heat also causes the hair to straighten so that the hair will maintain a straightened configuration after the crosslinking reaction.

Heat can be applied to the hair using various techniques. In one embodiment, for instance, a hair iron may be used to supply heat. In another embodiment, on the other hand, a blow dryer can be used to provide heat alone or in combination with combing.

Of particular advantage, the hair treatment composition of the present disclosure crosslinks with hair at temperatures as low as 40° C. to 80° C. For instance, the crosslinking reaction can occur at a temperature of less than about 200° C., such as at a temperature of less than about 180° C., such as at a temperature of less than about 170° C., such as at a temperature of less than about 160° C. In general, the temperature should be at least 40° C., such as at least 60° C., such as at least 70° C., such as at least 80° C.

The invention will now be further defined with reference to the accompanying examples.

### EXAMPLE 1

A composition comprising the following was prepared and adjusted to pH4.5 (using sodium hydroxide or citric acid as applicable):

| | |
|---|---|
| Water | 98.5% |
| Acetoacetylated Diethylene Glycol (as defined below) | 1.5% |

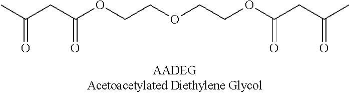 [SMILES: CC(=O)CC(=O)OCCOCCOC(=O)CC(C)=O]

A 2.5 g tress of level 9 bleached light brown hair was treated with the aforementioned composition as follows.

a) Soaked the tress in 25 ml solution of the composition at 35° C. for 10 minutes

b) Excess liquor was squeezed off

c) Dry the hair tress using a hair drier.

The hair tress was then evaluated by Differential Scanning Calorimetry (Perkin Elmer DSC6000). Samples (˜5 mg) were cut and placed into a stainless steel pan and 50 microlitres deionised Water was added before sealing. The heating profile was between 90-180° C. at 4° C. per minute.

A significant increase in denaturation temperature (Td) was observed on the “treated” sample when compared to hair treated with water alone. It has been reported in literature that an increase in Td may indicate an increase in crosslinking within the keratin fibres.

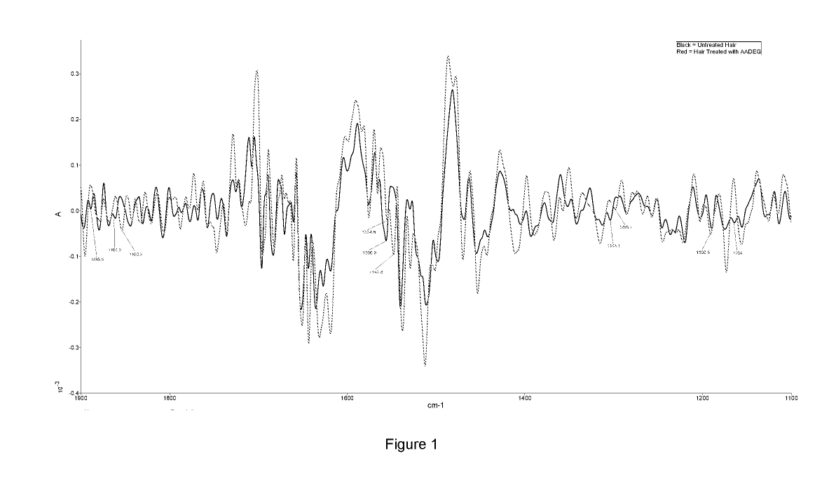

The same hair tresses were also analysed by Fourier Transform Infrared Spectroscopy (FTIR) (Perkin Elmer Frontier) using an UATR attachment. The resultant 2^{nd} derivative spectra (FIG. 1) show a number of new bonds when compared to untreated samples (bonds that are not present in the AADEG material itself). For example, in the FTIR spectra, the peaks at 1853 cm⁻¹ and 1547 cm⁻¹ are peaks which are neither present in the AADEG itself or the untreated hair, and conversely peaks at 1888 cm⁻¹ and 1164 cm⁻¹ are present in the untreated hair, but not in the treated hair.

This further indicates that an interaction between the AADEG and hair fibre has occurred and further substantiates the effects observed under DSC analysis.

### EXAMPLE 2

A composition comprising the following was prepared and adjusted to pH4.5 (using sodium hydroxide or citric acid as applicable):

| | |
|---|---|
| Water | 98.5% |
| Acetoacetylated Ethylene Glycol (as defined below) | 1.5% |

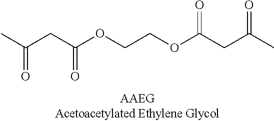 [SMILES: CC(=O)CC(=O)OCCOC(=O)CC(C)=O]

A 2.5 g tress of level 9 bleached light brown hair was treated with the aforementioned composition as follows.

a) Soaked the tress in 25 ml solution of the composition at 35° C. for 10 minutes

b) Excess liquor was squeezed off

c) Dry the hair tress using a hair drier.

The hair tress was then evaluated by Differential Scanning Calorimetry (Perkin Elmer DSC6000). Samples (˜5 mg) were cut and placed into a stainless steel pan and 50 microlitres deionised Water was added before sealing. The heating profile was between 90-180° C. at 4° C. per minute.

A significant increase in denaturation temperature (Td) was observed on the “treated” sample when compared to hair treated with water alone. It has been reported in literature that an increase in Td may indicate an increase in crosslinking within the keratin fibres.

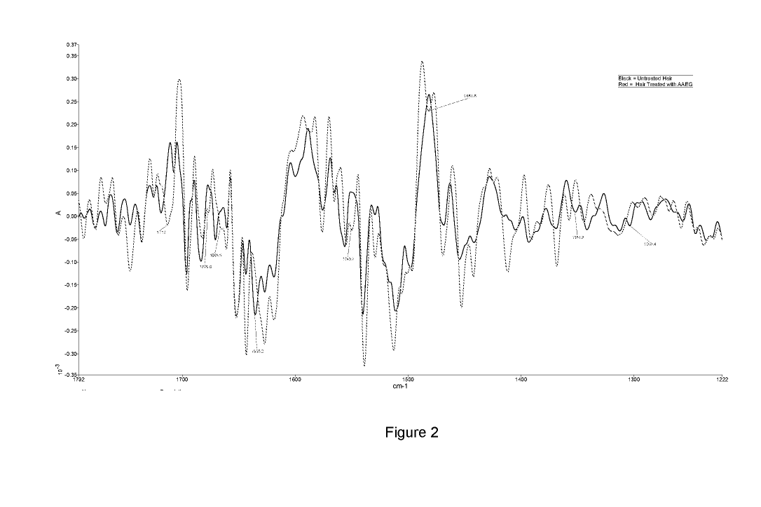

The same hair tresses were also analysed by Fourier Transform Infrared Spectroscopy (FTIR) (Perkin Elmer Frontier) using an UATR attachment. The resultant 2^{nd} derivative spectra (FIG. 2) show a number of new bonds when compared to untreated samples (bonds that are not present in the AAEG material itself). For example, in the FTIR spectra, the peaks at 1713 cm⁻¹ and 1481 cm⁻¹ are peaks which are neither present in the AADEG itself or the untreated hair, and conversely peaks at 1351 cm⁻¹ and 1303 cm⁻¹ are present in the untreated hair, but not in the treated hair.

This further indicates that an interaction between the AAEG and hair fibre has occurred and further substantiates the effects observed under DSC analysis.

### EXAMPLE 3

A composition comprising the following was prepared and adjusted to pH4.5 (using sodium hydroxide or citric acid as applicable):

| | |
|---|---|
| Water | 98.5% |
| Acetoacetylated Triethylene Glycol (as defined below) | 1.5% |

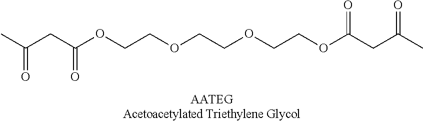 [SMILES: CC(=O)CC(=O)OCCOCCOCCOC(=O)CC(C)=O]

A 2.5 g tress of level 9 bleached light brown hair was treated with the aforementioned composition as follows.

a) Soaked the tress in 25 ml solution of the composition at 35° C. for 10 minutes

b) Excess liquor was squeezed off

c) Dry the hair tress using a hair drier.

The hair tress was then evaluated by Differential Scanning Calorimetry (Perkin Elmer DSC6000). Samples (˜5 mg) were cut and placed into a stainless steel pan and 50 microlitres deionised Water was added before sealing. The heating profile was between 90-180° C. at 4° C. per minute.

A significant increase in denaturation temperature (Td) was observed on the “treated” sample when compared to hair treated with water alone. It has been reported in literature that an increase in Td may indicate an increase in crosslinking within the keratin fibres.

### EXAMPLE 4

A composition comprising the following was prepared and adjusted to pH4.5 (using sodium hydroxide or citric acid as applicable):

| | |
|---|---|
| Water | 98.5% |
| Acetoacetylated Isosorbide (as defined below) | 1.5% |

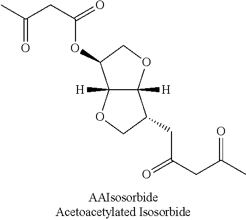 [SMILES: CC(=O)CC(=O)C[C@@H]1CO[C@H]2[C@@H]1OC[C@@H]2OC(=O)CC(C)=O]

A 2.5 g tress of level 9 bleached light brown hair was treated with the aforementioned composition as follows.

a) Soaked the tress in 25 ml solution of the composition at 35° C. for 10 minutes

b) Excess liquor was squeezed off

c) Dry the hair tress using a hair drier.

The hair tress was then evaluated by Differential Scanning Calorimetry (Perkin Elmer DSC6000). Samples (˜5 mg) were cut and placed into a stainless steel pan and 50 microlitres deionised Water was added before sealing. The heating profile was between 90-180° C. at 4° C. per minute.

A significant increase in denaturation temperature (Td) was observed on the “treated” sample when compared to hair treated with water alone. It has been reported in literature that an increase in Td may indicate an increase in crosslinking within the keratin fibres.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A hair treatment composition comprising an acetoacetylated compound.

2. A hair treatment composition as defined in claim 1, wherein the acetoacetylated compound is capable of crosslinking with NH₂ and/or OH groups.

3. A hair treatment composition as defined in claim 1, wherein the acetoacetylated compound comprises the following formula: 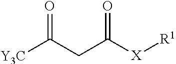
wherein Y comprises hydrogen or a halogen; X comprises oxygen, nitrogen or sulfur; and R¹ comprises one or more acetoacetylated groups, one or more thiol groups, one or more carbonyl groups, one or more hydroxyl groups, one or more vinyl groups, one or more epoxy groups, one or more acetal groups, or mixtures thereof.

4. A hair treatment composition as defined in claim 1, wherein the acetoacetylated compound comprises a poly(acetoacetylated) compound.

5. A hair treatment composition as defined in claim 1, wherein the acetoacetylated compound comprises the following formula: 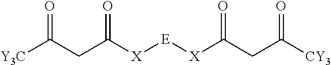
wherein X comprises nitrogen, oxygen or sulfur; Y comprises hydrogen or a halogen; and E comprises an alkylene group.

6. A hair treatment composition as defined in claim 1, wherein the acetoacetylated compound comprises an acetoacetylated polyol or an acetoacetamide.

7. A hair treatment composition as defined in claim 1, wherein the acetoacetylated compound comprises one of the following formulas: 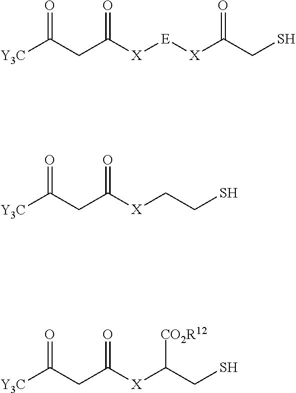
wherein X comprises nitrogen, oxygen or sulfur; Y comprises hydrogen or a halogen; E comprises an alkylene group; and R¹² comprises a C₁ to C₆ alkyl group.

8. A hair treatment composition as defined in claim 1, wherein the acetoacetylated compound comprises one of the following formulas: 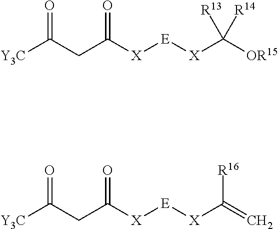
wherein X comprises nitrogen, oxygen or sulfur; Y comprises hydrogen or a halogen; E comprises an alkylene group; and wherein R¹³, R¹⁴, and R¹⁶ independently comprise hydrogen or a C₁ to C₄ alkyl group; and R¹⁵ is an acetyl group or a C₁ to C₄ alkyl group.

9. A hair treatment composition as defined in claim 1, wherein the acetoacetylated compound comprises one of the following formulas: 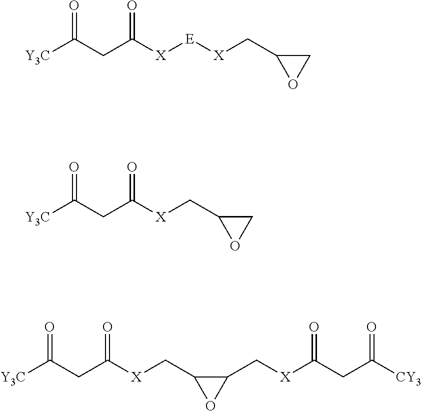
wherein X comprises nitrogen, oxygen or sulfur; Y comprises hydrogen or a halogen; and E comprises an alkylene group.

10. A hair treatment composition as defined in claim 1, wherein the composition further comprises a liquid carrier comprising water, an alcohol or mixtures thereof.

11. A hair treatment composition as defined in claim 10, further comprising a surfactant and a viscosity modifier.

12. A hair treatment composition as defined in claim 11, wherein the viscosity modifier comprises a polyol.

13. A hair treatment composition as defined in claim 1, further comprising a liquid carrier, a surfactant, a viscosity modifier, and a preservative, the acetoacetylated compound being present in the composition in an amount from about 0.1% to about 25% by weight.

14. A process for treating hair comprising:
contacting hair with a hair treatment composition comprising an acetoacetylated compound; and
applying sufficient heat to the treated hair so that the acetoacetylated compound causes hair fibers to crosslink.

15. A process as defined in claim 14, wherein heat is applied such that crosslinking occurs at a temperature of from about 40° C. to about 250° C.

16. A process as defined in claim 14, wherein heat is applied by contacting the treated hair with a straightening iron.

17. A process as defined in claim 14, wherein heat is applied by contacting the treated hair with heated air.

18. A process as defined in claim 14, wherein the acetoacetylated compound comprises a bis acetoacetylated compound.

19. A process as defined in claim 14, wherein the acetoacetylated compound comprises the following formula: 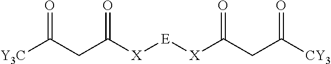
wherein X comprises nitrogen, oxygen or sulfur; Y comprises hydrogen or a halogen; and E comprises an alkylene group.

20. A process as defined in claim 14, wherein the process straightens the hair fibers.
